(19) United States
(12) Patent Application Publication
(10) Pub. No.: US 2023/0227394 A1
(43) Pub. Date: Jul. 20, 2023

(54) PROCESS FOR MINIMISING THE LOSS OF ACTIVITY IN REACTION STEPS CARRIED OUT IN CIRCULATION

(71) Applicant: Röhm GmbH, Darmstadt (DE)

(72) Inventors: Steffen KRILL, Muehltal (DE); Melanie Raczek, Darmstadt (DE); Rudolf Burghardt, Darmstadt (DE)

(73) Assignee: Röhm GmbH, Darmstadt (DE)

(21) Appl. No.: 18/006,958

(22) PCT Filed: Jul. 1, 2021
(86) PCT No.: PCT/EP2021/068155

§ 371 Date: Jan. 26, 2023

(30) Foreign Application Priority Data
Jul. 30, 2020 (EP) 20188528.2

(51) Int. Cl. C07C 51/235

(52) U.S. Cl. CPC C07C 51/235

(57) ABSTRACT

A novel process can be used for preparing methacrylates, such as methacrylic acid and/or alkyl methacrylates, especially MMA. The process allows for prolonging of the catalyst service life and an increase in efficiency of the methacrylate preparation based on C2 or C4 raw materials, especially when proceeding from isobutylene or tort-butanol or ethylene as raw material. The process allows for performance for longer periods without disruption, with constant or even increased activities and selectivities. This gives rise to the possibility of performing such processes in a very simple, economically viable, and environmentally benign manner. In addition, it has been possible to minimize known safety risks that emanate from the methacrolein intermediate.

### FIELD OF THE INVENTION

The present invention relates to a procedure for minimization of loss of activity in reaction steps performed in circulating operation. In numerous processes in the chemical industry, such a circulating mode of operation is employed when only a portion of the raw material supplied to the reactor is converted to the target product in a reaction step, and the remaining residue of the raw material, downstream of the reactor, is returned to the reactor inlet. The aim of such a partial conversion may be, for example, to minimize the irreversible formation of by-products that are formed to an increased degree in the region of high conversions, and, in the case of exothermic reactions, form as a result of the elevated reaction temperatures that occur at elevated conversions. The formation of such by-products increases the specific raw material costs of the desired product, and entails costs for the disposal of the by-products.

### PRIOR ART

The circulation mode can achieve the effect that the target product can be obtained in high yield from the raw material without suffering as a result of the by-products that typically occur in the region of high conversion.

For this purpose, in general, the raw material unconverted in the reaction step is separated from the target product by a suitable process step and, optionally together with other components likewise separated from the target product, fed back to the reactor input stream. Such process steps may include, but are not limited to, extractions, crystallizations, distillations, condensations and membrane separation methods. A general process scheme is shown in FIG. 1.

The unconverted raw material separated off in this way is frequently stored intermediately in a suitable apparatus before being fed back to the reactor input stream. Such apparatuses include, but are not limited to, gasometers, pressure vessels and storage tanks. The advantage of such a storage is that the concentration of the unconverted raw material in the reactor input stream can easily be kept constant by balancing out any fluctuations in the mass flows of the unconverted raw material that result from variable conversion levels. This is advantageous especially when the unconverted raw material streams from multiple identical reaction steps performed in parallel are to be collected in a common apparatus and fed back in the same distribution to all reactors. Making the input concentration of unconverted raw material constant avoids the adjustment of operating parameters, for example pressure and temperature, in the reaction step and hence assures reliable and undisrupted operation of production.

Such an apparatus for collection is typically designed such that the time before there is any need to adjust the operating parameters is as long as possible. Ideally, for example, an intermediate storage tank is designed with a large volume, such that constant recycling of unconverted raw material is possible even over a long period of time. In addition, it is advantageous when parameters such as pressure and temperature during intermediate storage are chosen to be as close as possible to the parameters of the input stream to the reaction step. It is thus possible to avoid energy-intensive compression, heating or cooling steps. In this context, for example, the intermediate storage of unconverted raw materials at a temperature similar to the input temperature of the reaction step is energetically very favourable.

Examples of such reaction steps executed in circulating operation include, but are not limited to, partial liquid phase hydrogenation, partial gas phase oxidation and oxidative esterification of unsaturated carbonyl compounds. Typically, such reactions are accelerated by catalysts, which may also be solid-state catalysts. In this case, these may, for example, also be part of a multistage reaction cascade in which the target product from a first reaction step is the raw material for a second reaction step. In addition, the second reaction step may also be executed in circulating operation, and the target product from the first reaction step may likewise be stored intermediately as raw material in a suitable apparatus for the second reaction step.

An important example of such a multistage reaction cascade is a process for two-stage oxidation of C4 species such as isobutene (I BEN) or tert-butanol (TBA) in the presence of water and air for preparation of methacrylic acid via the intermediate of methacrolein.

Methacrylic acid (MAA), an unsaturated carboxylic acid, is used as a starting material for production of plastics. The salts and esters of methacrylic acid are referred to as methacrylates. Among these, a known and important representative is methyl methacrylate (MMA).

More than 3 million tonnes of MAA are produced annually, which serve mainly as starting material for the synthesis of other chemical compounds. MAA can be obtained industrially proceeding from ethene (C2 species) or isobutene (C4 species), which are steamcracker products, via the intermediate of methacrolein (MAL).

MAL itself is used as intermediate for preparation of polymers, especially, for example, polymethylmethacrylate [PMMA], resins, crop protection products and, to a small degree, for flavourings and odourants.

In the preparation of methacrylates from ethylene, a C2 species, propionaldehyde obtained by hydroformylation from ethylene is first reacted in a first reaction step with formaldehyde to give MAL. In a second reaction step, this MAL can then be oxidatively esterified catalytically with methanol to give MMA. A corresponding process is described in WO 2014/170223. Alternatively, this MAL can also be oxidized to MAA, and optionally then esterified with methanol to give MMA.

In the preparation of MMA from C4 species, in general, IBEN or TBA or mixtures of IBEN and TBA are first converted in a first reaction stage in the gas phase together with an oxygen-containing gas mixture over a heterogeneous catalyst (“catalyst 1”) to give an MAL-containing gas mixture. The conversion of the C4 species here is virtually quantitative, and is reported, for example, to be greater than 99%. Processes for oxidation of C4 species to MAL are described, for example, in DE 10 2006 015710 or EP 19 952 32. The process gas phase leaving the first reaction stage is mixed with a cooler, recycled MAL-containing gas phase together with atmospheric oxygen and water vapour. This results in the feed gas for the second stage.

An MAL-containing gas mixture prepared in this way is then converted in a second reaction stage in the gas phase over a further heterogeneous catalyst (“catalyst 2”) to an MAA-containing gas mixture.

The second oxidation stage, like the first, is operated at a moderate gauge pressure between 0.1 and 2 bar and at temperatures between 260 and 360° C. For this purpose, heteropolyacid catalysts based on molybdenum and phosphorus and a few further dopants are used (see, for example, US2007/0010394). The modified heteropolyacids tend to achieve significantly poorer selectivities at higher conversions. For that reason, the conversion and the associated catalyst loading is set between 65% and 85%. For all processes and modifications thereof, this circumstance results in the need to separate unconverted methacrolein from the desired methacrylic acid product in the process gas and ultimately to return it upstream of the second oxidation reactor as recycled MAL.

Such a separation of methacrolein is typically performed by at least one distillation or extraction step. MAL itself is absorbed from the process gas and processed further, purified to some degree, and isolated by a desorption process and optionally at least one distillation operation. The absorption and desorption are optionally followed by a further distillation. As a result of the presence of volatile but condensable components in addition to acrolein, according to the separation complexity, crude methacrolein is obtained with some organic by-product constituents, usually an MAL mixture having an MAL concentration greater than 70% by weight. For example, GB 2004886 describes the passage of an MAL-containing reaction mixture through a quench column in which high-boiling components such as methacrylic acid are condensed. The MAL remaining in the residual gas is subsequently absorbed and, after stripping, fed in gaseous form to the second oxidation stage.

The methacrolein-containing mixture separated from the methacrylic acid, after the second reaction stage, according to the catalyst quality and parameters of the process regime, as well as methacrolein, also contains further by-products, including aldehydes. As prior art, the following by-product spectrum can be defined for the recycled MAL:

| | |
|---|---|
| 0.5-4% by weight of | acetaldehyde |
| 1-8% by weight of | acetone |
| 1-5% by weight of | acrolein |
| 0.05-0.4% by weight of | butane-2,3-dione |
| 0.2-1.5% by weight of | MMA |
| 1-5% by weight of | water |
| 1-5% by weight of | methacrylic acid |
| 0.1-3% by weight of | acetic acid |
| and hence | |
| 70-95% by weight of | methacrolein. |

A characteristic feature of the recycled MAL, according to the process, for example in a tandem operation of the first and second oxidation stages or intermediate isolation of MAL, is a methacrolein content greater than 70% by weight and the presence both of lower-boiling components such as acetone, acrolein and acetaldehyde and of higher-boiling components such as MMA, water and methacrylic acid.

The MAA obtained after the second reaction stage is subsequently converted to MMA with methanol. Comprehensive details of the C4-based method are given, inter alia, in Ullmann's Encyclopedia of Industrial Chemistry 2012, Wiley-VCH Verlag GmbH & Co. KGaA, Weinheim, Methacrylic Acid and Derivatives, DOI: 10.1002/14356007.a16_441. pub2, and in Krill and Rühling et al. “Viele Wege führen zum Methacrylsäuremethylester” [Many Routes lead to Methyl Methacrylate], WILEY-VCH Verlag GmbH & Co. KGaA, Weinheim, doi.org/10.1002/ciuz.201900869.

Overall, the following three routes for C4-based MMA production are well known:

Process A, “tandem C4 direct oxidation process (Sumitomo process)”, without intermediate isolation of methacrolein: Here, in a first step, MAL is prepared from isobutene and is oxidized in a process step 2 to MAA, before, in a process step 3, MMA is obtained by esterification of MAA with methanol. This process is also referred to in the literature as the “tandem process”, since the process gas from the first stage is oxidized directly to MAA without isolating the MAL intermediate.

Process B, “separate C4 direct oxidation (Mitsubishi process)”: Here, similarly to process A, MAL is prepared in a first process step from isobutene and is isolated and purified in liquid form in a separate process step, before being evaporated and oxidized to MAA in a process step 3, and finally converted to MMA by esterification in a process step 4.

Process C, “direct metha process (Asahi process)” or direct oxidative esterification process: Here too, in a first process step, MAL is prepared in the gas phase over a first catalyst from isobutene and is likewise first isolated and intermediately purified in a process step 2, before being directly oxidatively esterified to MMA in a process step 3. Process step 3 is conducted in the liquid phase over a suspended catalyst. Thus, the combination of a gas phase step and a process step in the liquid phase is the essential difference in this process compared to processes A and B, in which the two partial oxidations are conducted over different catalysts, each in the gas phase.

All the processes described are well documented in the prior art, for example in (i) IHS Chemical Process Economics Program, Review 2014-05, R. J. Chang, Syed Naqvi or (ii) S. Nakamura, H. Ichihashi, Vapor Phase Catalytic Oxidation of Isobutene to Methacrylic Acid, Stud. Surf. Sci. Catal. 1981, 7, 755-767.

The partial oxidation of MAL to MAA in method A or B proceeds exothermically and can be accelerated by the use of heteropolyacid (HPA) catalysts as “catalyst 2”. For this purpose, a gas mixture—also referred to as feed—consisting of air, water vapour, unconverted recycled reaction gas, and unconverted recycled MAL is oxidized over catalyst 2. The reactor used for this purpose is typically in the form of a shell and tube apparatus, and the heat released is removed via a salt bath executed in one or more zones. As described in EP 0006248, catalyst 2 may comprise formulations including molybdenum, vanadium, phosphorus and oxygen.

While the oxidation of MAL to MAA in the second reaction stage can be performed with a fresh catalyst, even in the case of comparatively low salt bath temperatures of, for example, T=260 to 300° C., it is necessary to increase the salt bath temperature over the course of the catalyst lifetime, for example to 310 to 330° C., in order to compensate for ageing-related loss of catalyst activity. The increase in the salt bath temperature keeps the conversion and the MAA yield based on a single pass through the catalyst bed constant, which is a prerequisite for the operation of the process within an economically viable range.

The increase in the salt bath temperature also results in a continuous rise in the reaction gas temperature. Toward the end of the catalyst lifetime, which is typically 1 to 3 years according to the gas loading, expressed as GHSV (“gas hourly space velocity”), the reaction gas temperature reaches a critical range in which there can be autocatalytic reaction within the oxygen- and methacrolein-containing reaction mixture, called the “post-combustion” phenomenon. The post-combustion phenomenon is also referred to as silent oxidation or the blue flame phenomenon. This describes processes in which thermally sensitive organic substrate molecules, for example methacrolein or other aldehydes, in gaseous oxygen-containing reaction mixtures, even at temperatures below the nominal ignition temperature, break down into shorter-chain fragments of lower molar mass at a reduced rate compared to explosion events, which is accompanied by a spontaneous increase in pressure and temperature of the reaction mixture. These spontaneous pressure and temperature events need not necessarily lead to failure of vessels or apparatuses, but can cause deformation of construction elements, damage to measurement devices or triggering of safety devices, such as bursting discs, and hence force an interruption to the operation of the plant.

In addition, a post-combustion phenomenon can also function as ignition source for explosive self-ignition of the reaction mixture. In the case of self-ignition, there is a very significant rise in the temperature in the reaction gas between the tube base exit of the second oxidation reactor and the downstream separation apparatus, for example a quench column, which can lead to an interruption in production, for example via a safety switch. The higher the temperature of the reaction gas, the more common are post-combustion phenomena and hence also instances of self-ignition. Typically, the occurrence of the post-combustion phenomenon thus indicates that the catalyst is reaching the end of its life.

The rate at which the catalyst ages, i.e. suffers a decrease in its activity, increases with rising temperature. Raising the salt bath temperature thus has the effect that a time-consuming and costly exchange of the oxidation catalyst has to be conducted more frequently than if the temperature is kept constant. The oxidation reactor is consequently operated at a temperature that permits assurance of economic reliable operation of the oxidation reaction on the one hand, and limiting of catalyst ageing to a minimum on the other hand. This prior art gives rise to various problems that are in need of a technical solution. For instance, as well as a temperature-related decrease in activity of catalyst 2, activity can also be reduced by damaging accompanying substances in the feed to the downstream reaction stages.

One example of such an accompanying substance is IBEN that has not been fully converted in the first reaction stage. The IBEN in the feed for the second reaction stage can be minimized by maintaining a high, virtually quantitative conversion in the first reaction stage. As described in Nagai et al., Sumitomo Kagaku 2004, 1-12, increasing IBEN tolerance of catalysts for the oxidation of MAL is a constant field of work in catalyst research.

A second example of a compound that has an adverse effect on activity is 2-methyl pentenal, which can form as a by-product, for example, in the preparation of MAL from propanal and formaldehyde (DE 3508702).

A further example of an accompanying substance that impairs the activity of catalyst 2 is dimeric MAL, or simply di-MAL, which is formed from MAL by Diels-Alder reaction:

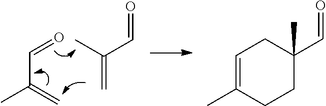

EP 0194620 describes the adverse effect of di-MAL on the activity of a catalyst 2. Accordingly, the MAA yield achieved in the 2nd reaction stage over a catalyst of the Mo₁₂V_{0.6}P_{1.2}Cs₂Cu_{0.7}Rh_{0.1}Oₓ type is reduced by 5% when the feed contains 0.4% by mass of di-MAL.

It is known that the lifetime of a catalyst used for the second oxidation stage can be increased by keeping the concentration of the components mentioned in the feed low. For instance, DE 3508702 describes a process in which the sum total of the concentration of 2-methylpentenal and di-MAL in the feed is lower than 0.15% by weight, and the concentration of propanal and formaldehyde is lower than 1.0% by weight. However, the publication does not give any indications as to how compliance with these upper concentration limits can be achieved economically in operation.

DE 2143582 describes the suppression of the formation of oligomeric and polymeric products in the high-temperature storage of liquid methacrolein, for example 5 hours at 100° C., when the methacrolein has been stabilized with a mixture of 500 ppm each of orthophosphoric acid and p-tert-butylcatechol. Disadvantages of this are firstly the large amount of stabilizer required, but secondly especially the presence of a free mineral acid that causes corrosion damage under the process conditions.

A further negative aspect of di-MAL formation in the storage of methacrolein relates to the fact that the di-MAL formed in the intermediate storage is oxidized in the second oxidation stage to give high-boiling compounds. This di-MAL is removed in the subsequent workup steps and is thus no longer available for recovery of methacrylic acid. U.S. Pat. No. 2,577,445 describes a process for redissociation of di-MAL to MAL. In this process, di-MAL is first converted to its semicarbazone, and the latter is then cleaved pyrolytically to give MAL. A disadvantage of the method mentioned is the introduction of a further process step.

A process for suppression of di-MAL formation in the intermediate storage of MAL is described in JP 2006028067. According to this, the formation of di-MAL can be avoided by storing MAL at 0° C. in a storage vessel in the presence of 1000 ppm of hydroquinone. The storage vessel here is filled only to an extent of 70% of its volume with MAL, and the gas phase in the headspace in the remaining 30% of the container volume must be enriched with 6% to 16% by volume of oxygen for maintenance of the ability of the hydroquinone stabilizer to function. The process described has the crucial disadvantage of a high safety-related risk as a result of the formation of a mixture containing oxygen and combustible substances in the headspace of the storage vessel. The risk is aggravated especially when there is an increase in the MAL vapour pressure in the event of failure of the cooling of the storage vessel. What is not described, moreover, is the effect of the high hydroquinone content on the activity in the downstream reaction steps.

According to the above remarks, there is thus a challenge in ensuring a low concentration of the accompanying substances that have an adverse effect on the oxidation of methacrolein to methacrylic acid in the methacrolein feed to the downstream reaction stage. More particularly, it is necessary to ensure that the formation of di-MAL during the intermediate storage is suppressed.

### Problem

The problem addressed by the present invention was thus that of providing a novel process for preparing alkyl methacrylates and optionally methacrylic acid—especially MMA—which, proceeding from C2 or C4 units, can be operated without interruption over a long period of time.

A further problem addressed by the present invention was that of obtaining alkyl methacrylates and optionally methacrylic acid by oxidation of methacrolein arising from C2 or C4 sources, wherein the catalysts used for the oxidation undergo reduced ageing as a result of a suitable process regime with incompletely converted raw materials and intermediates.

A further problem optionally addressed by the present invention was that of obtaining alkyl methacrylates, especially methyl methacrylate, by direct oxidative esterification of methacrolein arising from C2 or C4 sources, wherein the alkyl methacrylates have a low content of free methacrolein.

It was a further problem addressed by the present invention to minimize the operating and safety-related risks that emanate from the raw materials and intermediates incompletely converted in the oxidation of methacrolein.

It was a particular object of the present invention to provide a process in which MAL unconverted after oxidation of the methacrolein intermediate to methacrylic acid is separated off and recovered in a suitable form, and at the same time the formation of by-products that have an adverse effect on the downstream process steps can be reduced to a concentration level that is not detrimental to these process steps.

Further problems that are not mentioned explicitly may be apparent from the claims and the description of the invention that follows, without being set out explicitly.

### Solution

The problems mentioned are solved by a novel process for preparing alkyl methacrylates and optionally methacrylic acid, especially methyl methacrylate (MMA), from C2 or C4 raw materials, especially in a process with methacrolein as intermediate. It is a feature of this process of the invention that the storage temperature and the storage volume and, as a result, the storage time of methacrolein can be kept low.

C4 raw materials used may be substances such as isobutene (IBEN) or methyl tert-butyl ether (MTBE). MTBE is a commonly and widely used raw material for C4-based processes. It can be easily transported and permits the operation of methacrylic acid or alkyl methacrylate production even at a relatively high distance from C4 sources, for example steamcrackers. IBEN can be obtained from C4 streams by etherification with methanol to give MTBE and subsequent redissociation. C2-based processes proceed from ethylene, which is first converted to propanal and finally reacted with formaldehyde to give methacrolein.

The process of the invention is based on a process in which, in a first reaction stage in a first reactor—e.g. reactor A or J—a methacrolein-containing fraction is prepared from a C4 source f2 or a C2 source f6. In addition, a second methacrolein-containing fraction based on the first fraction is then converted further in at least one second reactor. This process is characterized in that the methacrolein-containing second fraction in liquid form is kept in cooled storage in an intermediate vessel D to a storage temperature between −30° C. and 50° C. with a dwell time of less than 48 h, and thence is guided into an evaporator E or in liquid form into a second reactor, which is a reactor G for an oxidative esterification.

The recycling of the predominantly methacrolein-containing stream 3b into the input stream upstream of reactor B is accomplished by, in accordance with the invention, first feeding the liquefied stream to an intermediate vessel D and storing it therein. It is known that reaction products of higher molar mass can form in the course of storage of liquid methacrolein-containing substance mixtures, and these have an adverse effect on the catalytic activity of subsequent reaction steps (see, for example, DE 3508702). One example of a representative of these compounds is the cyclic dimer of methacrolein (di-MAL), which originates from MAL via Diels-Alder reaction. As described in the prior art, the formation of components such as di-MAL can be suppressed by measures involving either the addition of large amounts of stabilizers, some of them corrosive (see, for example, DE 2143582), or storage under an oxygen-containing atmosphere (see, for example, JP 2006028067). While additional amounts of stabilizer can be deposited in the pipelines and apparatuses of the process, the combination of large amounts of methacrolein and oxygen-containing gas phases in storage vessels entails an incalculable safety risk.

It has now been found that, surprisingly, the formation of by-products that adversely affect the downstream reaction steps can be prevented in a simple, technically robust, economically viable manner, and at low risk to safety, by cooled storage of methacrolein in the storage in the intermediate vessel D only in small amounts and hence with a short dwell time. In addition, it is possible to achieve a stable mode of process operation, which is usually assisted by the use of large buffer volumes for intermediates, specifically by the minimization of such buffer volumes.

Storage for a short time at reduced temperature suppresses the formation of by-products, especially of di-MAL. Preference is given to processes in which the condensation temperature of the methacrolein-containing stream in the separation apparatus C is higher than the storage temperature of the methacrolein-containing stream in the intermediate vessel D. The invention includes those executions of the process in which the dwell time of the methacrolein-containing stream in the intermediate vessel D is less than 48 h and the storage temperature is between −30° C. and 50° C. Preference is given to those executions of the process in which the dwell time of the methacrolein-containing stream in the intermediate vessel D is less than 12 h, more preferably less than 6 h, and the storage temperature is between −20° C. and 30° C., more preferably between −10° C. and 20° C.

The inventive minimization of the storage volume and of the storage temperature for methacrolein, as well as the advantages described, is associated with various favourable safety-related side effects that assist a reliable and accident-free process regime:

The first side effect relates to the high inhalation toxicity of methacrolein vapours in conjunction with the high vapour pressure of methacrolein. In the event of unintended release of methacrolein, the temperature dependence of the vapour pressure allows the radius of spread thereof to be minimized if the methacrolein is kept in cooled storage. The second side effect relates to the propensity of highly reactive compounds such as methacrolein to undergo self-accelerating reactions in the course of storage that can lead to an abrupt increase in pressure and temperature in the storage vessel and to resultant vessel failure. The damage that arises can likewise be kept low by the minimization of the storage volume, while the probability of occurrence of such an event is minimized in that the temperature is kept well below the self-accelerating polymerization temperature (SAPT) in the course of storage. The SAPT of a substance indicates that lower temperature above which a self-sustaining polymerization reaction can occur; it combines the effects of outside temperature, polymerization kinetics, vessel size and the specific removal of heat that occurs through the wall of the storage tank. The dependence of the SAPT on the removal of heat is shown for MAL in Table 1. It is naturally the case that the specific removal of heat rises together with rising surface/volume ratio as the vessel volume falls, which means that the storage temperature considered to be safe with regard to polymerization events rises as the volume of the storage tank falls.

TABLE 1
Self accelerating polymerization temperature of methacrolein
depending on the removal of heat in the storage tank

| Specific removal of heat [mW/(kg*K)] | SAPT [° C.] |
|---|---|
| 60 | 25 |
| 80 | 30 |
| 100 | 35 |
| 150 | 35 |
| 200 | 40 |

The third side effect relates to the fact that such self-accelerating reactions are typically suppressed by the addition of stabilizers. However, as a result of the circulation of unconverted methacrolein, such stabilizers are deposited in the form of solids that have to be removed from the production plant on shutdown at high cost and inconvenience. A low storage temperature permits the lowering of the stabilizer concentration and hence minimizes the deposits.

A further possible effect of the cooled intermediate storage of methacrolein for a short period of time in accordance with the invention results from the high reactivity of the di-MAL in downstream reaction steps.

It is additionally preferable that the predominantly methacrolein-containing stream of matter 3b which is obtained after workup and removal of the predominantly methacrylic acid-containing stream 3a is condensed and stored in such a way that the concentration of methacrolein dimers (di-MAL) in the liquid feed stream 4 to the evaporator E or—as described further down—to reactor G does not exceed a content of 1% by weight.

In a first embodiment of the invention, proceeding from a C4 source f2, in the first reactor A, a methacrolein-containing process gas 1 is formed as the first methacrolein-containing fraction. This is then converted in the form of a process gas 1, after optionally mixing in an oxygen- and water vapour-containing gas mixture 6, to a process gas 7 which is converted in a second reaction stage in a reactor B by partial oxidation in the gas phase to a methacrylic acid-containing process gas 2.

In reactor A, the C4 source is oxidized here to methacrolein at temperatures between 320 and more than 400° C. at slightly elevated pressure in the presence of atmospheric oxygen and water vapour, and optionally a recycle gas obtained in the later part of the workup. The conversion in the tandem process is greater than 98%. The dwell time in the shell and tube reactor with modern doped bismuth molybdate catalysts, as described in U.S. Pat. No. 5,929,275 for example, is typically 1 to 4 seconds. GHSV values between 1000 and 2000 s⁻¹ are attained. The exiting methacrolein-containing process gas 1 is mixed with a colder mixed stream 5 comprising a recycled MAL gas phase and the gas mixture 6 arising from an atmospheric oxygen-containing gas mixture f3 and a water vapour-containing gas mixture f4. This results in the feed gas 7 for the second oxidation stage. It is likewise optionally possible to introduce the atmospheric oxygen-containing gas mixture f3 and/or the water vapour-containing gas mixture f4 for evaporation of the methacrolein-containing stream 4 into the evaporator E. The second oxidation stage in reactor B, like the first, is operated at a moderate gauge pressure between 0.1 and 2 bar and at temperatures between 260 and 360° C. For this purpose, heteropolyacid catalysts based on molybdenum and phosphorus and a few further dopants are used (as described, for example, in US 2007/0010394). The modified heteropolyacids have increasingly poorer selectivities for methacrylic acid at higher conversions. For that reason, the conversion and the associated catalyst loading is preferably set between 65% and 85%. For all C4-based processes, and also for this embodiment of the process according to the invention, this circumstance results in the need to separate unconverted MAL from the desired methacrylic acid product in the resulting process gas 2 downstream of reactor B and ultimately to return it upstream of the second oxidation reactor B as recycled MAL.

In preferred executions of this embodiment, this is followed by these steps:
a. The process gas 2 is separated in a separation apparatus C into a predominantly methacrylic acid-containing stream 3a and a predominantly methacrolein-containing stream 3b.
b. The separation apparatus described in step a includes at least one quenching step, one crystallization step and one distillation step.
c. The predominantly methacrolein-containing stream 3b is condensed at the top of a fractional distillation column and the condensed methacrolein-containing stream 3b is then guided into the intermediate vessel D.
d. A thermostatted, especially cooled, methacrolein-containing stream 4 is then guided out of intermediate vessel D into the evaporator E in order to convert stream 4, optionally with involvement of an atmospheric oxygen-containing gas mixture f3 and/or a water vapour-containing gas mixture f4, to a methacrolein-containing gas stream 5 therein, which is guided into reactor B together with gas mixture 6, optionally mixed to give a gas stream 7.

The process gas formed in the second reaction stage in reactor B can then be separated in a downstream separation apparatus C. A separation is effected here into a predominantly methacrylic acid-containing stream 3a and a predominantly methacrolein-containing stream 3b. The separation is necessary in order to convert the portion of the methacrolein unconverted in reactor B to the desired methacrylic acid target product as completely as possible by recycling upstream of the reactor. The separation processes underlying the separation apparatus C include at least one quenching step, one crystallization step and one distillation step, and the predominantly methacrolein-containing stream 3b is obtained by condensation at the top of a fractional distillation column.

The hot process gas 2 from reactor B typically leaves the reactor at 250 to 360° C. and first has to be cooled down. This typically involves firstly cooling to a temperature between 150 and 250° C. by means of a recuperative gas cooler. Recuperative gas coolers are preferred because these utilize the heat for steam generation. Accordingly, the gas phase, now with reduced temperature, is typically guided into a circulating condensed quench phase at temperatures between 50 and 100° C. This quench phase may be the bottoms fraction of a quench column which is circulated by means of a pump and thermostatted. At the top of this quench column, the majority of the methacrolein passes over in gaseous form together with the process gas, while the majority of the methacrylic acid formed is condensed and quenched in the bottom. In a next process step, methacrolein is preferably condensed or absorbed together with water. In this step, methacrolein is obtained in liquid form together with all condensable secondary components, for example low boilers. Nevertheless, effective separation from the process gas is achieved; the latter escapes at the top of this column. In a last step, methacrolein can now be desorbed from the absorber phase, thus giving a predominantly methacrolein-containing stream 3b having an MAL content of greater than 70% by weight.

A stream p1 containing methacrylic acid in pure form can be obtained after suitable workup from the predominantly methacrylic acid-containing stream 3a. This workup is more preferably effected by distillation and/or extraction.

In an alternative further embodiment of this first embodiment of the process according to the invention, the predominantly methacrylic acid-containing stream 3a is fed to a reactor F, which is especially an esterification reactor, where the methacrylic acid, preferably under acid catalysis, is converted together with a stream f8 containing a suitable alcohol to give an alkyl methacrylate-containing product stream p2. Corresponding process parameters for obtaining pure methacrylic acid or pure alkyl methacrylates can be taken, for example, from EP 19 952 32.

A cooled, predominantly methacrolein-containing stream of matter 4 obtained in this way, in this first embodiment, is preferably fed back to the oxidation process by first evaporating it in an evaporator E to give a gas stream 5. Optionally, this gas stream 5 can be combined with a gas mixture 6 arising from an oxygen-containing gas stream f3 and a water vapour-containing gas stream f4 to give a process gas 7, in order then to mix it as such into the process gas 1 upstream of reactor B. Preference is given to those executions of the process of this embodiment in which the isobutene content in the process gas 1 does not exceed a value of 2000 ppm by volume, and workup, thermostatting or intermediate cooling and reevaporation of the methacrolein-containing process gas result in a methacrolein-containing stream 5 which, based on methacrolein, contains 0.2% to 25% by weight, preferably to 20% by weight, of further carbonylic C₁-C₄ hydrocarbons. It is further preferable that this process gas has a content of methacrolein dimers of less than 1% by weight. As a result of the low content of di-MAL in the methacrolein-containing stream 5 and hence also in the input stream to reactor B by virtue of small storage amounts and/or low storage temperatures, it is demonstrably possible to keep the reaction temperature in reactor B at a low level for longer. Bearing in mind the adverse effect of the reactor temperature on the lifetime of a methacrolein oxidation catalyst, it is thus surprisingly possible to achieve an extension in catalyst lifetime by storing intermediately stored MAL at minimum temperatures for minimum periods of time.

If the starting materials in the preparation of alkyl methacrylates are not C4 sources, it is possible, in a second, very preferred embodiment of the present invention, to perform the process according to the invention proceeding from a C2 source f6, preferably ethylene. Here, the propanal intermediate prepared from ethylene, for example, is converted to a purified, liquid methacrolein-containing stream 13.

More particularly, in this embodiment, proceeding from the C2 source f6 in a reactor H, by formation of a propionaldehyde-containing stream 8 which is worked up by means of a purification step I comprising at least one distillation, a propionaldehyde-containing stream 10 is formed. This stream is then converted in a downstream reactor J and a further downstream purification step K to a methacrolein-containing stream 13, and this methacrolein-containing stream 13 is finally guided into the intermediate vessel D.

Alternatively, stream 13 can also be converted directly to the gas phase in the evaporator E. In this variant, only the MAL from stream 3b is guided into the intermediate vessel D and stored therein.

For this purpose, first of all, ethylene is hydroformylated together with a synthesis gas stream f5 in a reactor H to give propanal. The hydroformylation reaction conducted for the conversion of ethylene to propanal is described in detail in the standard literature, for example in E. Billig, D. Bryant, Kirk-Othmer Encyclopedia of Chemical Technology, John Wiley & Sons, Inc., OXO Process and R. Franke et al., Applied Hydroformylation, dx.doi.org/10.1021/cr3001803, Chem. Rev. 2012, 112, 5675-5732.

In general, catalysts are used for this reaction. The preferred catalysts especially include compounds comprising rhodium, iridium, palladium and/or cobalt, particular preference being given to rhodium. In a particular embodiment, it is especially possible to use complexes comprising at least one phosphorus compound as ligand for catalysis. Preferred phosphorus compounds include aromatic groups and at least one phosphorus atom, more preferably two phosphorus atoms. The phosphorus compounds especially include phosphines, phosphites, phosphinites, phosphonites. Examples of phosphines are triphenylphosphine, tris(p-tolyl)phosphine, tris(m-tolyl)phosphine, tris(o-tolyl)phosphine, tris(p-methoxyphenyl)phosphine, tris(p-dimethylaminophenyl)phosphine, tricyclohexylphosphine, tricyclopentylphosphine, triethylphosphine, tri-(1-naphthyl)phosphine, tribenzylphosphine, tri-n-butylphosphine, tri-t-butylphosphine. Examples of phosphites are trimethyl phosphite, triethyl phosphite, tri-n-propyl phosphite, tri-i-propyl phosphite, tri-n-butyl phosphite, tri-i-butyl phosphite, tri-t-butyl phosphite, tris(2-ethylhexyl) phosphite, triphenyl phosphite, tris(2,4-di-t-butylphenyl) phosphite, tris(2-t-butyl-4-methoxyphenyl) phosphite, tris(2-t-butyl-4-methylphenyl) phosphite, tris(p-cresyl) phosphite. Examples of phosphanites are methyldiethoxyphosphine, phenyldimethoxyphosphine, phenyldiphenoxyphosphine, 2-phenoxy-2H-dibenz[c,e][1,2]oxaphosphorin and derivatives thereof in which the hydrogen atoms have been wholly or partly replaced by alkyl and/or aryl radicals or halogen atoms. Standard phosphinite ligands are diphenyl(phenoxy)phosphine and its derivatives diphenyl(methoxy)phosphine and diphenyl(ethoxy)phosphine.

Catalysts and ligands for hydroformylation are detailed, for example, in WO 2010/030339 A1, WO 2008/071508 A1, EP 982314 B1, WO 2008/012128 A1, WO 2008/006633 A1, WO 2007/036424 A1, WO 2007/028660 A1, WO 2005/090276 A1, with reference being made to these publications for disclosure purposes, and the catalysts and ligands disclosed therein being encompassed by this application. These publications also detail reaction conditions. For hydroformylation of ethene, carbon monoxide and hydrogen are typically used in the form of a mixture, called synthesis gas. The composition of the synthesis gas used for hydroformylation may vary within wide ranges. The molar ratio of carbon monoxide and hydrogen is generally 2:1 to 1:2, especially about 45:55 to 50:50. The temperature in the hydroformylation reaction is generally within a range from about 50 to 200° C., preferably about 60 to 190° C., especially about 90 to 190° C. The reaction is preferably performed at a pressure in the range from about 5 to 700 bar, preferably 10 to 200 bar, especially 15 to 60 bar. The reaction pressure may be varied depending on the activity of the hydroformylation catalyst used. Suitable pressure-resistant reaction apparatuses for hydroformylation are known to those skilled in the art. These include the generally customary reactors for gas-liquid reactions, for example gas circulation reactors, bubble columns etc., which may optionally be divided by internals. Further preferred configurations of a hydroformylation reaction are set out in EP 12 946 68 inter alia, the contents of this publication being incorporated into the present application by reference.

After the hydroformylation step, the liquid, predominantly propionaldehyde-containing process stream 8, in a separation apparatus I, is separated into a liquid process stream 9 containing predominantly the hydroformylation catalyst and a purified liquid propionaldehyde-containing stream 10, with the liquid process stream 9 containing predominantly the hydroformylation catalyst being returned back to the hydroformylation reactor H. Suitable embodiments of such a separation apparatus are described, for example, in WO 2007/036424 A1 or EP 12 946 68.

The purified liquid propionaldehyde-containing stream 10 is then fed to a reactor J, where it is converted together with a formalin-containing stream f7 in a Mannich-like reaction to a liquid, predominantly methacrolein-containing process stream 11. Preferred processes for preparing methacrolein proceeding from propanal and formalin are described inter alia in publications U.S. Pat. No. 7,141,702, DE 3213681, U.S. Pat. Nos. 4,408,079, 2,848,499; JPH 04173757 A, JP 3069420 B2 and EP 03 179 09.

The reaction of propanal and formaldehyde is performed in the presence of organic bases, preferably amines and simultaneously acids, where the acids are generally inorganic acids. Examples of these are organic mono-, di- or polycarboxylic acid, preferably monocarboxylic acid, especially aliphatic monocarboxylic acid. Carboxylic acids used are appropriately aliphatic monocarboxylic acids having 1 to 10, preferably 2 to 4, carbon atoms, or di- and polycarboxylic acids having 2 to 10, preferably 2 and 4 to 6, carbon atoms. The dicarboxylic acids and polycarboxylic acids may be aromatic, araliphatic and preferably aliphatic carboxylic acids. Suitable examples are acetic acid, propionic acid, methoxyacetic acid, n-butyric acid, isobutyric acid, oxalic acid, succinic acid, tartaric acid, glutaric acid, adipic acid, maleic acid, fumaric acid. Other organic acids are likewise usable in principle, but are generally less appropriate for reasons of cost. Inorganic acids used are generally sulfuric acid and phosphoric acid. It is also possible to use acid mixtures. For the reaction of propanal and formaldehyde, particular preference is given to using at least one organic acid, more preferably acetic acid. The proportion of acid is between 0.1 and 20 mol %, advantageously from 0.5 to 10 mol %, preferably 1 to 5 mol %, based on propanal.

Preferred organic bases are especially secondary amines. Use for amines of this kind preferably include amines of the formula R₁R₂NH in which R₁ and R₂ are the same or different and may each denote an alkyl radical having 1 to 10, advantageously 1 to 8, especially 1 to 4 carbon atoms, which may also be substituted by ether groups, hydroxyl groups, secondary and tertiary amine groups, especially by 1 or 2 of these groups, an aralkyl radical having 7 to 12 carbon atoms, a cycloalkyl radical having 5 to 7 carbon atoms, R1 and R2 together with the adjacent nitrogen may also be members of a heterocyclic, advantageously 5- to 7-membered ring that may also contain a further nitrogen atom and/or an oxygen atom and may be substituted by hydroxyalkyl or alkyl groups having 1 to 4 carbon atoms. Suitable amines are, for example: dimethylamine, diethylamine, methylethylamine, methylpropylamine, dipropylamine, dibutylamine, di-isopropylamine, di-isobutylamine, methylisopropylamine, methylisobutylamine, methyl-sec-butylamine, methyl(2-methylpentyl)amine, methyl(2-ethylhexyl)amine, pyrrolidine, piperidine, morpholine, N-methylpiperazine, N-hydroxyethylpiperazine, piperazine, hexamethyleneimine, diethanolamine, methylethanolamine, methylcyclohexylamine, methylcyclopentylamine, dicyclohexylamine or corresponding mixtures. It may further be the case that at least one of the amines used does not have a hydroxyl group. More preferably, the proportion of amines having at least one hydroxyl group is not more than 50% by weight, preferably not more than 30% by weight and more preferably not more than 10% by weight, based on the weight of the amines used. The proportion of organic base, preferably secondary amines, is between 0.1 and 20 mol %, advantageously from 0.5 to 10 mol %, preferably 1 to 5 mol %, based on propanal. The ratio of equivalents of amine to acid is preferably chosen so as to result in a pH of 2.5 to 9 in the reaction mixture before the reaction. It may further be the case that the molar ratio of acid to organic base, preferably amine, is in the range from 20:1 to 1:20, preferably in the range from 10:1 to 1:10, more preferably in the range from 5:1 to 1:5 and especially preferably in the range from 2:1 to 1:2.

The reaction temperature for the reaction of propanal with formaldehyde at the exit of the reaction zone is between 100 and 300° C., preferably 130 and 250° C., preferably from 140 to 220° C., especially 150 to 210° C. The reaction pressure is in the range from 2 to 300, preferably 5 to 250, more preferably from 10 to 200 bar, advantageously from 15 to 150 bar, preferably 20 to 100 bar and especially 40 to 80 bar. Pressure and temperature are adjusted such that the reaction is always effected below the boiling point of the reaction mixture, i.e. the reaction proceeds in the liquid phase. All pressure figures in the context of the present application are given as absolute pressure in the measurement unit of bar. The dwell time or reaction time is preferably not more than 25 minutes, appropriately 0.01 to 25 minutes, advantageously 0.015 to 10 minutes, preferably 0.03 to 2 minutes. More preferably, the dwell time or reaction time is in the range from 0.1 to 300 seconds, especially preferably in the range from 1 to 30 seconds. The reactor used in the case of dwell times below 10 minutes is advantageously a tubular reactor. The dwell time is based here on the time over which the reaction mixture is converted. All components are present here at reaction pressure and temperature, and so this time can be calculated from the distance between mixing point and the expansion point. The expansion point is the point at which the mixture is brought from the reaction pressure to a pressure below 5 bar. As well as water, the reaction mixtures may additionally include organic solvents, for example propanol, dioxane, tetrahydrofuran, methoxyethanol.

It may additionally be the case that the reaction of propanal with formaldehyde to give methacrolein in reactor J is effected in the presence of preferably at least 0.1% by weight, more preferably at least 0.2% by weight and especially preferably at least 0.5% by weight of methanol, based on formalin. In spite of these higher methanol concentrations, on account of the reaction regime according to the invention, for the optional subsequent step G of direct oxidative esterification, it is possible to dispense with a costly and inconvenient removal of methanol at the formalin preparation and/or methacrolein purification stage. In a particular configuration, formaldehyde and propanal may be mixed before these reactants are brought to reaction pressure and/or temperature.

The reaction can be performed in reactor J as follows: A mixture of propanal, amine, formaldehyde and appropriately water and/or acid and/or base is kept at the reaction temperature and the reaction pressure during the reaction time. In a preferred embodiment, a mixture (appropriately equimolar mixture) of formaldehyde and propanal can be heated to the desired reaction temperature by means of a heat exchanger and fed to a tubular reactor. A catalyst solution (solution of the secondary amine and an acid, appropriately in H₂O) can be injected into this mixture at the reactor inlet, which is optionally likewise heated to the reaction temperature by means of a heat exchanger. The highly exothermic reaction sets in, and the reaction mixture is heated further. The pressure under which the reaction proceeds is preferably kept at such values, by means of a pressure-retaining valve at the outlet of reactor J, that the reaction mixture still remains liquid even at high temperatures in the reactor during the reaction time. After the reaction, the reaction mixture can be expanded to standard pressure and worked up in the separation apparatus K. In the preparation of methacrolein from propanal and formaldehyde, the reaction mixture is preferably fed to a column and stripped therein with steam. The methacrolein leaves the column overhead together with water. The mixture is condensed and separated by means of a phase separation vessel into an upper phase and a lower phase. The upper phase is guided as the purified liquid methacrolein-containing stream into the above-described intermediate vessel D, or alternatively into the above-described evaporator E. The lower phase consists mainly of water. It can preferably be recycled at least partly back into the column for removal of the methacrolein still dissolved therein. The aqueous catalyst solution can be drawn off at the bottom of the column together with the water formed in the reaction and the water from the formaldehyde solution. For the further processing, when very little amine and/or acid is used and it is therefore no longer worth recycling the catalyst, the bottoms liquid can be discarded. In the case of greater amine and/or acid concentrations in the bottoms discharge, it is alternatively possible to remove in part water by distillation and to recycle it back into the reactor as a liquid process stream 12 containing predominantly the Mannich catalyst system. It is also possible to divide the bottoms output into two substreams such that one substream carries exactly the amount of water which has been formed in the reaction and introduced with the starting materials. This substream is then discharged and the remaining proportion is recycled into the reactor. Aqueous formaldehyde and propanal can also be preheated separately and fed to the reactor.

In a particularly preferred embodiment, methacrolein can be prepared from propanal and formaldehyde in a tandem reaction, wherein propanal is obtained by the reaction of ethylene, carbon monoxide and hydrogen according to steps H and I and reacted directly with formaldehyde according to steps J and K. This process is described in detail by Deshpande et al., Biphasic catalysis for a selective oxo-Mannich tandem synthesis of methacrolein, Journal of Molecular Catalysis A: Chemical 211 (2004) 49-53, doi:10.1016/j.molcata.2003.10.010 and in U.S. Pat. No. 7,141,702.

In addition, there are two further embodiments of the present invention, the third embodiment being a variation of the first embodiment in which the C4-based MAL is not fed to the evaporator E for later gas phase oxidation to methacrylic acid but guided into a reactor G where oxidative esterification is effected.

Analogously, the fourth embodiment relates to MAL based on the preparation in the first part of the second embodiment from C2 raw materials.

In these two further embodiments of the process according to the invention, the cooled, predominantly MAL-containing stream 14 obtained after the intermediate vessel D may be fed to a reactor G, where it is subjected to a direct oxidative esterification (DOE) reaction with a stream f9 containing an alcohol and an oxygen-containing gas mixture f10. The DOE reaction proceeds in the liquid phase and gives rise to the product stream p3 containing the corresponding alkyl methacrylate from MAL and the alcohol in one reaction step. The di-MAL present in the MAL feed likewise contains a carbonyl group and can therefore likewise be converted to the corresponding alkyl ester.

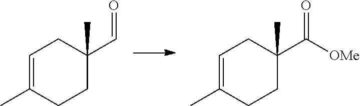

It has been found that di-MAL has a higher reactivity than MAL in the DOE reaction and hence is converted preferentially to the corresponding alkyl ester. A suitable workup sequence for alkyl methacrylate obtained via DOE first envisages the distillative removal of the lower-boiling excess alcohol compared to the alkyl methacrylate at lower temperature, and then the distillative separation of the alkyl methacrylate from higher-boiling by-products at higher temperature (see, for example, WO 2014 170223). In the first distillation step, di-MAL having a boiling point of 171° C. and di-MAL methyl ester having a boiling point of 204° C. thus remain together with MMA having a boiling point of 100° C. in the bottom product and are distilled over together in the second separation step, while excess methanol (boiling point of 65° C.) is removed overhead. In the second separation step performed at higher temperature, the di-MAL alkyl ester present in the alkyl methacrylate can be cleaved by the thermal stress to give one equivalent of MAL and one equivalent of alkyl methacrylate. Di-MAL unconverted in the DOE reaction is cleaved in the same way to give two equivalents of MAL. There is thus the risk of obtaining an MAL-contaminated alkyl methacrylate in this way. EP 34 504 22 discloses that, for example, the presence of free methacrolein in MMA adversely affects the polymerization properties of MMA. A further advantage of the short-term and cold intermediate storage of MAL according to the invention is thus that the process of polymerization of MMA obtained via DOE from MAL stored in this way is facilitated. It should also be noted that di-MAL itself, and the methyl ester formed therefrom, can lead to unwanted yellow colours in the end product, especially in PMMA.

The examples that follow document the suppression of formation of by-products by suitable storage of methacrolein and the favourable effect of low-by-product methacrolein on the performance of the methacrolein oxidation catalyst. Further examples demonstrate the conversion of the di-MAL by-product to di-MAL methyl ester in the direct oxidative esterification (DOE) of MAL to MMA, and hence the transport of high-boiling MAL equivalents into the workup sequence of an MMA DOE process.

1.

### LIST OF REFERENCE SYMBOLS

### Description of the Figures

FIG. 1 shows a process scheme of preferred embodiments of the process according to the invention for conversion of C2 or C4 sources to methacrylic acid or alkyl methacrylates.

FIG. 2 shows the progression of the di-MAL concentrations in MAL over time as a function of storage time and temperature.

FIG. 3 shows an enlarged section from FIG. 2.

FIG. 4 shows the summary of the oil bath temperatures observed in Examples 2 to 4 in oxidation of methacrolein (c[di-MAL_{fresh}]=35 ppm) that was stored before the start of the experiment at temperature T_{L} for duration of t_{L}.

### APPARATUSES

A First oxidation reactor
B Second oxidation reactor
C Separation apparatus
D Intermediate vessel
E Evaporator
F Esterification reactor
G Direct oxidative esterification
H Propionaldehyde synthesis reactor
I Propionaldehyde catalyst removal
J Methacrolein synthesis reactor
K Methacrolein catalyst removal
L Mixing point 1
M Mixing point 2

### STREAMS

1 Methacrolein-containing process gas from the first oxidation reactor
2 Methacrylic acid-containing process gas from the second oxidation reactor
3a Predominantly methacrylic acid-containing stream from separation apparatus C
3b Predominantly methacrolein-containing stream from separation apparatus C
4 Cooled, predominantly methacrolein-containing stream in the feed stream to evaporator E
5 Methacrolein-containing gas stream
6 Oxygen- and water vapour-containing gas mixture
7 Methacrolein-containing process gas feed to the second oxidation reactor
8 Liquid, predominantly propionaldehyde-containing process stream
9 Liquid, predominantly hydroformylation catalyst-containing process stream
10 Purified liquid propionaldehyde-containing stream
11 Liquid, predominantly methacrolein-containing process stream
12 Liquid process stream containing predominantly the Mannich catalyst system
13 Purified liquid methacrolein-containing stream
14 Cooled, predominantly methacrolein-containing stream in the feed stream to reactor G
f1 O₂-containing gas in the feed stream to reactor A
f2 C4 source in the feed stream to reactor A
f3 O₂-containing gas for mixing of the oxygen- and water vapour-containing gas mixture 6
f4 Water vapour for mixing of the oxygen- and water vapour-containing gas mixture 6
f5 Synthesis gas stream in the feed stream to reactor H
f6 C2 source in the feed stream to reactor H
f7 Formalin-containing stream in the feed stream to reactor J
f8 Alcohol-containing stream in the feed stream to reactor F
f9 Alcohol-containing stream in the feed stream to reactor G
f10 O₂-containing gas in the feed stream to reactor G
p1 Optionally purified methacrylic acid product stream from reactor C
p2 Optionally purified alkyl methacrylate product stream from reactor F
p3 Optionally purified alkyl methacrylate product stream from reactor G

### OTHER REFERENCE SYMBOLS

t(L) Storage time in intermediate vessel D
T(L) Storage temperature in intermediate vessel D
GC Gas chromatography
MAL Methacrolein
di-MAL Cyclic methacrolein dimer
ppm Parts per million
E_{A} Activation energy [kJ/mol]
A Impact factor [ppm/d]
c(i) Concentration of component i
GHSV Gas hourly space velocity
X(i) Conversion of component i [%]
S(i) Selectivity of component i [%]
Y(i) Yield of component i [%]
MAA Methacrylic acid
MMA Methyl methacrylate
T(oil) Oil bath temperature
T(35 cm) Reactor temperature within the catalyst bed at a distance of 35 cm from the reactor inlet
T(45 cm) Reactor temperature within the catalyst bed at a distance of 45 cm from the reactor inlet
SAPT Self accelerating polymerization temperature [° C.]
B.p. Boiling point [° C.]
DOE Direct oxidative esterification
PMMA Poly(methylmethacrylate)
IBEN Isobutene
TBA tert-Butyl alcohol
MTBE Methyl tert-butyl ether

### DESCRIPTION OF THE FIGURES

With regard to the figures, it should be noted that further components known to the person skilled in the art may additionally be present for performance of the process according to the invention. For example, in general, each of the columns listed will have a condenser. It should also be noted that not every preferred embodiment is included in the drawings. The position of the feed lines additionally does not indicate their real position, but merely illustrates the topological arrangement of the corresponding process steps.

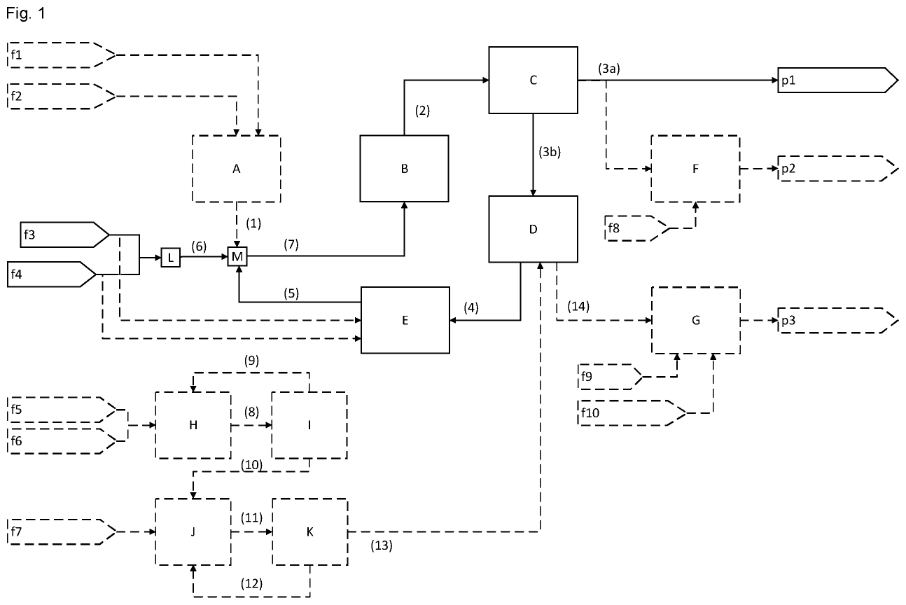

FIG. 1 shows the general process scheme of the reaction of C2 or C4 sources with an oxygen- and water vapour-containing gas to give methacrylic acid or alkyl methacrylates. In one variant, a C4 source f2 is converted together with an oxygen-containing gas f1 in a reactor A to a methacrolein-containing process gas 1 which, after a further methacrolein-containing process gas 5 and optionally a gas stream 6 arising from an oxygen-containing stream f3 and a water vapour-containing stream f4 has been mixed in, gives rise to a methacrolein-containing process stream 7 which is fed to a reactor B. In reactor B, the methacrolein-containing process stream 7 is converted to a methacrylic acid-containing process stream 2 which, in the subsequent separation apparatus C, is separated into a preferably methacrylic acid-containing stream 3a and a preferably methacrolein-containing process stream 3b. After optional purification steps, a methacrylic acid-containing product stream p1 can be obtained directly from the preferably methacrylic acid-containing stream 3a. Alternatively, the preferably methacrylic acid-containing process stream 3a can be converted together with an alcohol-containing stream f8 in a reactor F to a product stream p2 containing alkyl methacrylates. The preferably methacrolein-containing process stream 3b is stored in liquid form in a cooled intermediate vessel D and fed as cooled process stream 4 to an evaporator E, where it is converted to the above-described methacrolein-containing process gas 5 which, as set out above, is fed back upstream of the inlet of reactor B.

Optionally, the liquid, preferably methacrolein-containing process stream 14 can be converted together with an oxygen-containing gas mixture f10 and an alcohol-containing stream f9 in a reactor G in a direct oxidative esterification reaction directly to a product stream p3 containing alkyl methacrylates.

In a further, alternative variant, a C2 source f6 is converted together with synthesis gas f5 and a catalyst in a hydroformylation reactor H to a propionaldehyde-containing process stream 8 which, in a separation apparatus I, is separated into a preferably catalyst-containing process stream 9 and a low-catalyst, propionaldehyde-containing stream 10. While the preferably catalyst-containing process stream 9 is fed back to the hydroformylation reactor H, the low-catalyst, propionaldehyde-containing stream 10 is converted together with a formalin-containing stream f7 and a further catalyst in reactor J to a methacrolein-containing stream 11 which, in the downstream separation apparatus K, is separated into a preferably catalyst-containing process stream 12 and a preferably methacrolein-containing liquid process stream 13. The preferably methacrolein-containing liquid process stream 13 may, in this process variant, be fed directly to the evaporator E already described, where it is converted together with the cooled, preferably methacrolein-containing process stream 4 to the methacrolein-containing process gas 5. Alternatively, the preferably methacrolein-containing liquid process stream 13 can first be stored together with the preferably methacrolein-containing process stream 3b already described in the cooled intermediate vessel D likewise already described to form the cooled process stream 4. The methacrolein-containing process gas 5 obtainable in both alternatives can be converted as described above to methacrylic acid or alkyl methacrylates.

### EXAMPLES

### Example 1 (Kinetics of Formation of di-MAL from MAL)

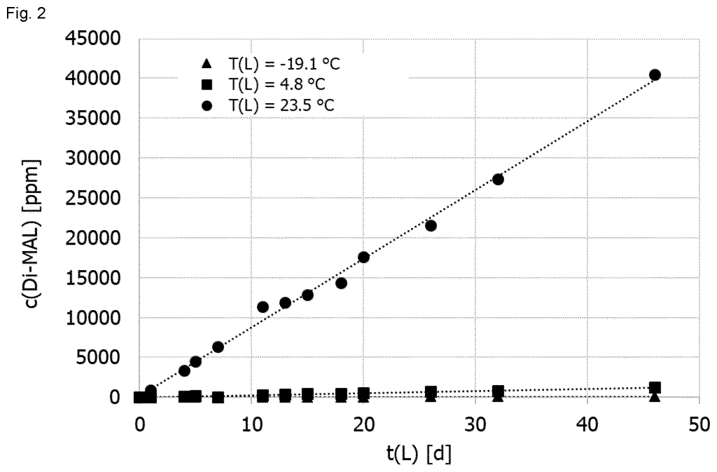

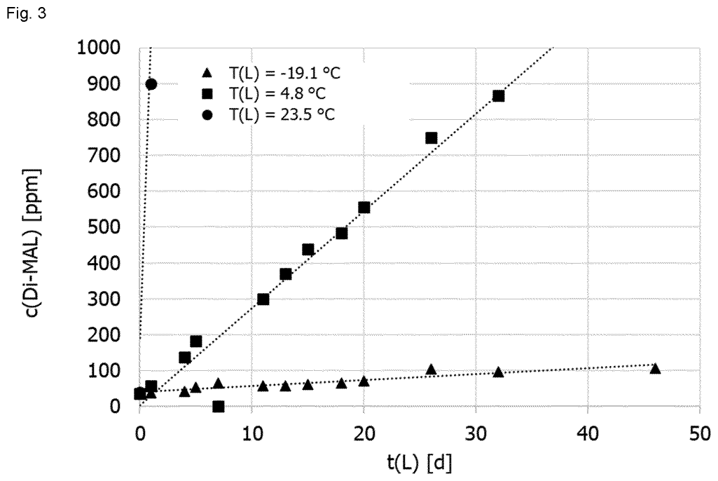

For the study of the formation kinetics of di-MAL from MAL, MAL was stored at different temperatures for 46 days, and the di-MAL concentration was monitored continuously by means of GC. The MAL used for the experiments was distilled beforehand in order to obtain a minimum di-MAL starting concentration (35 ppm here). The di-MAL concentrations ascertained, which are summarized in Table 2 and in FIG. 2 and FIG. 3, can be used to derive pseudo-zeroth-order kinetics of di-MAL formation with the following formation law (E_{A}=90.25 kJ/mol, A=4.57*10¹⁸ ppm/d):

$$c ( t ) = {c}_{0} + ( A * {e}^{\frac{- {E}_{A}}{R * T}} ) * t$$

At a storage temperature of −5° C., formation of 12 ppm of MAL per day can be expected. At 25° C., by contrast, nearly 700 ppm of MAL per day is formed, and at 50° C. 1.2%/d.

TABLE 2
Concentration of di-MAL in MAL as a function
of storage temperature T(L) and time t(L)

| | | c(di-Mal) (ppm) | | |
|---|---|---|---|---|
| Sample | T(L) [d] | T(L) = −19.1° C. | T(L) = 4.8° C. | T(L) = 23.5° C. |
| 0 | 0 | 35.35 | 35.35 | 40 |
| 1 | 1 | 38.47 | 56.79 | 900 |
| 2 | 4 | 41.64 | 138.06 | 3400 |
| 3 | 5 | 53.95 | 183.27 | 4500 |
| 4 | 7 | 64.81 | — | 6400 |
| 5 | 11 | 58.2 | 300.14 | 11400 |
| 6 | 13 | 58.05 | 370.8 | 11900 |
| 7 | 15 | 60.69 | 438.89 | 12900 |
| 8 | 18 | 64.8 | 484.05 | 14400 |
| 9 | 20 | 70.25 | 554.75 | 17600 |
| 10 | 26 | 104.33 | 749.8 | 21600 |
| 11 | 32 | 96.35 | 865.74 | 27400 |
| 12 | 46 | 106.27 | 1239.89 | 40500 |

### Comparative Example 1 (Gas Phase Oxidation of MAL; c(Di-MAL)=10 000 ppm)

The gas phase oxidation of MAL to MAA was studied in a continuously operated tubular reactor. For monitoring of the reaction temperature, the reactor is equipped with 2 thermocouples at a distance of 35 cm and 45 cm from the reactor inlet. For the experiment, methacrolein having a di-MAL content of 35 ppm is first stored at 25° C. for 10 days, after which it has a di-MAL content of 10 000 ppm. The MAL is converted to the gas phase in an evaporator at 160° C. with the aid of a gas stream composed of air and nitrogen. The gas mixture that results after further addition of air and nitrogen (MAL/O₂/H₂O/N₂=1:2.5:4.5:22.5) is passed over a molybdenum-bismuth mixed oxide catalyst at a GHSV of 1070 h⁻¹, in the course of which the temperature of the oil bath is adjusted so as to result in an MAL conversion X(MAL) of 65.8%. The MAL conversion is determined by comparing the gas compositions ascertained by means of GC on entry to and exit from the reactor. Once the conversion has stabilized after an operating time of 12 h, the oil bath temperature is raised to such an extent that an MAL conversion X(MAL) of 68.8% is attained. In the same way, conversions of 74.9% and 75.3% are established by further increasing the oil bath temperature. The oil bath temperatures (T[oil]) required and the methacrylic acid selectivities (S[MAA]) and temperatures observed within the catalyst bed at a distance of 35 cm and 45 cm from the reactor inlet are compiled in Table 3.

TABLE 3
Temperatures T, conversions X(MAL) and methacrylic
acid selectivities S(MAA) in the gas phase oxidation
of methacrolein (di-MAL content 10 000 ppm)

| | | | T(oil)/ | T (35 cm)/ | T (45 cm)/ |
|---|---|---|---|---|---|
| Example | X(MAL)/% | S(MAA)/% | ° C. | ° C. | ° C. |
| 2-1 | 65.8 | 89.7 | 288.5 | 307.1 | 308 |
| 2-2 | 68.8 | 89.3 | 289.8 | 312 | 311.2 |
| 2-3 | 74.9 | 88.6 | 293.7 | 323.2 | 319.7 |
| 2-4 | 75.3 | 89 | 293.7 | 322 | 317.7 |

### Example 2 (Gas Phase Oxidation of MAL; c(Di-MAL)=5000 ppm; Effect of Shorter Storage Time)

The gas phase oxidation of MAL to MAA was studied analogously to Comparative Example 1. For the experiment, methacrolein having a di-MAL content of 35 ppm is first stored at 25° C. for 5 days, after which it has a di-MAL content of 5000 ppm. The gas mixture that results after evaporation of MAL and mixing-in of nitrogen and air, as described in Comparative Example 1, is passed over the catalyst, in the course of which the temperature of the oil bath is adjusted so as to result in an MAL conversion of 66.3%. Once the conversion has stabilized after an operating time of 12 h, the oil bath temperature is raised to such an extent that an MAL conversion X(MAL) of 67.8% is attained. In the same way, conversions of 70.1% and 75.4% are established by further increasing the oil bath temperature. The oil bath temperatures (T[oil]) required and the methacrylic acid selectivities (S[MAA]) and temperatures observed within the catalyst bed at a distance of 35 cm and 45 cm from the reactor inlet are compiled in Table 4.

TABLE 4
Temperatures, conversions and methacrylic acid selectivities in
the gas phase oxidation of methacrolein (di-MAL content 5000 ppm)

| | | | T(oil)/ | T (35 cm)/ | T (45 cm)/ |
|---|---|---|---|---|---|
| Example | X(MAL)/% | S(MAA)/% | ° C. | ° C. | ° C. |
| 3-1 | 66.3 | 88.5 | 287.4 | 311.4 | 317.1 |
| 3-2 | 67.8 | 87.8 | 287.4 | 311.4 | 317.1 |
| 3-3 | 70.1 | 87.7 | 289 | 315.6 | 320.5 |
| 3-4 | 75.4 | 87.6 | 291.7 | 321.6 | 319 |

### Example 3 (Gas Phase Oxidation of MAL; Di-MAL Content 300 ppm; Effect of Lower Storage Temperature)

The gas phase oxidation of MAL to MAA was studied analogously to Comparative Example 1. For the experiment, methacrolein having a di-MAL content of 35 ppm is first stored at 5° C. for 10 days, after which it has a di-MAL content of 300 ppm. The gas mixture that results after evaporation of MAL and mixing-in of nitrogen and air, as described in Comparative Example 1, is passed over the catalyst, in the course of which the temperature of the oil bath is adjusted so as to result in an MAL conversion of 64.0%. Once the conversion has stabilized after an operating time of 12 h, the oil bath temperature is raised to such an extent that an MAL conversion of 65.5% is attained. In the same way, conversions of 69.0%, 71.5%, 74.3%, 74.9% and 76.4% are established by further increasing the oil bath temperature. The oil bath temperatures (T[oil]) required and the methacrylic acid selectivities (S[MAA]) and temperatures observed within the catalyst bed at a distance of 35 cm and 45 cm from the reactor inlet are compiled in Table 5.

TABLE 5
Temperatures, conversions and methacrylic acid selectivities in
the gas phase oxidation of methacrolein (di-MAL content 300 ppm)

| | | | T(oil)/ | T (35 cm)/ | T (45 cm)/ |
|---|---|---|---|---|---|
| Example | X(MAL)/% | S(MAA)/% | ° C. | ° C. | ° C. |
| 4-1 | 64 | 89.6 | 285.1 | 303.4 | 318.4 |
| 4-2 | 65.5 | 88.5 | 285.2 | 308.6 | 312.7 |
| 4-3 | 69 | 86.8 | 287.4 | 308.4 | 323.3 |
| 4-4 | 71.5 | 87.3 | 287.4 | 316.3 | 318.5 |
| 4-5 | 74.3 | 84.2 | 290.4 | 321.4 | 324.4 |
| 4-6 | 74.9 | 83 | 290.3 | 314.3 | 336.6 |
| 4-7 | 76.4 | 88.6 | 290.9 | 320.7 | 313.4 |

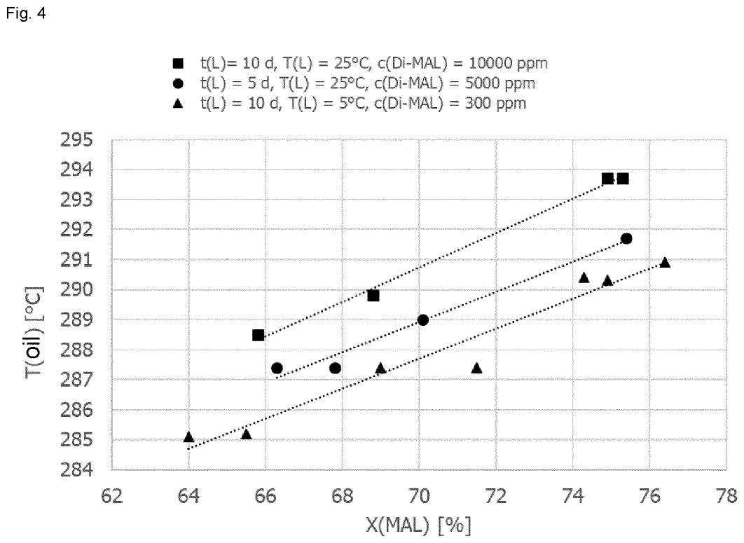

The oil bath temperatures observed in Comparative Example 1 and in Examples 2 and 3 are summarized in FIG. 4. It becomes clear that, for the entire conversion range examined, that MAL which requires the highest oil bath temperature was stored at the highest temperature (25° C.) for the longest time (10 d). By halving the storage time, the oil bath temperature can be lowered by an average of 2 K. By reducing the storage temperature from 25° C. to 5° C. with the same storage time, the oil bath temperature can even be lowered by an average of 3 K. Bearing in mind the adverse effect of the bath temperature on the lifetime of a methacrolein oxidation catalyst, it is thus possible to achieve an extension in catalyst lifetime by storing intermediately stored MAL at minimum temperatures for minimum periods of time.

### Comparative Example 2 (Direct Oxidative Esterification of MAL in the Liquid Phase in a Batch Test; c(Di-MAL)=10 000 ppm)

For the experiment, methacrolein having a di-MAL content of 35 ppm is first stored at 25° C. for 10 days, after which it has a di-MAL content of 10 000 ppm. The MAL pretreated in this way (1.2 g) is suspended together with an AuCoO/SiO₂—Al₂O₃—MgO catalyst (384 mg) and methanol (9.48 g) in a 140 ml steel autoclave with a magnetic stirrer. The pH of the MAL was first adjusted to 7.0 with 1% NaOH in MeOH, and the MAL was stabilized with 100 ppm of Tempol. The autoclave was pressurized to 30 bar gauge with a gas mixture of 7% O₂ in N₂. The explosion limit of the mixture is 8% by volume of oxygen. The autoclave was heated to 80° C. for 2 hours, cooled down and degassed, and the suspension was filtered. The filtrate was analysed by means of GC. The conversion of MAL was 64.0%, the selectivity for MMA was 93.7%, and the space-time yield was 10.6 mol of MMA/kg of catalyst per hour. The filtrate also contained 500 ppm of di-MAL and 10 450 ppm of di-MAL ester. It is found that 95% of the di-MAL present in the feed is oxidatively esterified to di-MAL methyl ester, and about 5% is unchanged in the reaction.

### Example 4 (Direct Oxidative Esterification of MAL in the Liquid Phase in a Batch Test; c(Di-MAL)=300 ppm; Effect of Low Storage Temperature)

The direct oxidative esterification of MAL to MMA was studied analogously to Comparative Example 2. For the experiment, methacrolein having a di-MAL content of 35 ppm is first stored at 5° C. for 10 days, after which it has a di-MAL content of 300 ppm. The MAL pretreated in this way (1.2 g) is suspended together with an AuCoO/SiO₂—Al₂O₃—MgO catalyst (384 mg) and methanol (9.48 g) in a 140 ml steel autoclave with a magnetic stirrer. The pH of the MAL was first adjusted to 7.0 with 1% NaOH in MeOH, and the MAL was stabilized with 100 ppm of Tempol. The autoclave was pressurized to 30 bar gauge with a gas mixture of 7% O₂ in N₂. The explosion limit of the mixture is 8% by volume of oxygen. The autoclave was heated to 80° C. for 2 hours, cooled down and degassed, and the suspension was filtered. The filtrate was analysed by means of GC. The conversion of MAL was 67.0%, the selectivity for MMA was 93.7%, and the space-time yield was 11.1 mol of MMA/kg of catalyst per hour. The filtrate also contained 15 ppm of di-MAL and 315 ppm of di-MAL ester. It is found that 95% of the di-MAL present in the feed is oxidatively esterified to di-MAL methyl ester, and about 5% is unchanged in the reaction. Compared to Example 5, it is additionally found that the reduced di-MAL content in the feed as a result of the low storage temperature was able to increase the space-time yield of the target reaction.

1: A process for preparing methacrylic acid and/or alkyl methacrylate, the process comprising:
forming a first methacrolein-containing fraction from a C2 source or a C4 source in a first reactor, and
reacting a second methacrolein-containing fraction, based on the first methacrolein-containing fraction, in at least one second reactor,
wherein the second methacrolein-containing fraction is in liquid form and is kept in cooled storage in an intermediate vessel to a storage temperature between −30° C. and 50° C. with a dwell time of less than 48 h, and thence is guided into an evaporator or in liquid form is guided into the at least one second reactor for an oxidative esterification.

2: The process according to claim 1, wherein, proceeding from the C4 source in the first reactor, a methacrolein-containing process gas is formed as the first methacrolein-containing fraction.

3: The process according to claim 1, wherein, proceeding from the C2 source in the first reactor, a first propionaldehyde-containing stream is formed which is worked up by a purification comprising at least one distillation, to form a second propionaldehyde-containing stream, which is in turn converted in a downstream reactor and a further downstream purification to a third methacrolein-containing stream, and this third methacrolein-containing stream is guided into the intermediate vessel.

4: The process according to claim 3, wherein a liquid methacrolein-containing stream is guided from the intermediate vessel into the evaporator, where the liquid methacrolein-containing stream is evaporated to form a methacrolein-containing process gas.

5: The process according to claim 2, wherein the methacrolein-containing process gas is guided together with an oxygen- and water vapour-containing gas mixture, optionally premixed as a gas stream, into the at least one second reactor, which is an oxidation reactor, forming a methacrylic acid-containing process gas in the at least one second reactor,
wherein
a. the methacrylic acid-containing process gas is separated in a separation apparatus into a predominantly methacrylic acid-containing stream and a predominantly methacrolein-containing stream,
b. the separation apparatus includes at least one quenching, one crystallization, and one distillation,
c. the predominantly methacrolein-containing stream is condensed at the top of a fractional distillation column and a condensed methacrolein-containing stream is then guided into the intermediate vessel, and
d. a thermostatted methacrolein-containing stream is then guided out of the intermediate vessel into the evaporator in order to convert the thermostatted methacrolein-containing stream to the methacrolein-containing gas stream therein, which is guided into the at least one second reactor together with the oxygen- and water vapour-containing gas mixture, optionally mixed to give the gas stream.

6: The process according to claim 5, wherein the predominantly methacrylic acid-containing stream is purified by distillation and/or extraction, and is optionally further reacted with an alcohol in a further reactor under acidic catalysis, to give the alkyl methacrylate.

7: The process according to claim 1, wherein a methacrolein-containing stream is taken in liquid form from the intermediate vessel and reacted in the at least one second reactor in the presence of an oxygen-containing gas, a catalyst, and an alkyl alcohol in the liquid phase in a direct oxidative esterification, to obtain an alkyl methacrylate-containing substance mixture.

8: The process according to claim 5, wherein, in the methacrolein-containing process gas, an isobutene content does not exceed a value of 2000 ppm by volume, and
wherein workup, thermostatting and re-evaporation of the thermostatted methacrolein-containing stream arising from the methacrolein-containing process gas give rise to a methacrolein-containing stream which, based on methacrolein, contains 0.2% to 25% by weight of further carbonylic C₁-C₄ hydrocarbons and has a content of methacrolein dimers (di-MAL) of less than 1% by weight.

9: The process according to claim 5, wherein the predominantly methacrolein-containing stream, which is obtained after workup and removal of the predominantly methacrylic acid-containing stream, is condensed and stored in such a way that a concentration of methacrolein dimers in the thermostatted methacrolein-containing stream to the evaporator or to the at least one second reactor does not exceed a content of 1% by weight.

10: The process according to claim 5, wherein a condensation temperature of the methacrylic acid-containing process gas is higher than a storage temperature of the condensed methacrolein-containing stream in the intermediate vessel.

11: The process according to claim 1, wherein the dwell time of the second methacrolein-containing stream in the intermediate vessel is less than 12 h and the storage temperature is between −20° C. and 30° C.

12: The process according to claim 11, wherein the dwell time in the intermediate vessel is less than 6 h and the storage temperature is between −10° C. and 20° C.
